(19) United States
(12) Patent Application Publication
(10) Pub. No.: US 2003/0171420 A1
(43) Pub. Date: Sep. 11, 2003

(54) Compounds and compositions for use in ingibting endoparasitic fatty acid biosynthesis

(76) Inventors: Colin Berry, Morganstown Cardiff (GB); John L. Harwood, Radyr Cardiff (GB)

Correspondence Address: MERCHANT & GOULD PC, P.O. BOX 2903, MINNEAPOLIS, MN, 55402-0903, US

(21) Appl. No.: 10/169,601

(22) PCT Filed: Jan. 8, 2001
(86) PCT No.: PCT/GB01/00082

(30) Foreign Application Priority Data
Jan. 6, 2000 (GB) 0000131.1

(51) Int. Cl. A61K 31/381

(52) U.S. Cl. 514/445

(57) ABSTRACT

Use of at least one compound, or pharmaceutically acceptable salt or prodrug thereof, as an inhibitor of at least one β-ketoacyl acyl carrier protein synthase operable in the fatty acid biosynthesis of endoparasites, which compound is of the general formula (I) where R₁ is selected from the group consisting of hydrogen, alkyl, (cyano)alkylene, alkenyl, alkynyl, (alkoxy)alkylene, (alkoxy)alkenylene, (alkoxy)alkynylene, cycloalkyl, (cycloalkyl)alkylene, (cycloalkyl)alkenylene, (cycloalkyl)alkynylene, (hetero-cycle)alkylene, (heterocycle)alkenylene, (heterocycle)alkynylene, aryl, (aryl)alkylene, (aryl)alkenylene, (aryl)alkynylene, (arylcarbonylarylene)alkylene, (arylcarbonylarylene)alkenylene and (arylcarbonylarylene)alkynylene; R₂ is alkyl or cycloalkyl; R₃ is alkyl or cycloalkyl; and R₄ is hydrogen or alkyl; including racemic mixtures and enantiomers of said compound when the latter is chiral, but excluding the racemic mixture of a chiral compound of formula (I) in which R₁ is CH₂═CH—C(CH₃)═CH—, R₂ is methyl, R₃ is methyl and R₄ is hydrogen.

The present invention is concerned with compounds and compositions for use in inhibiting endoparasitic fatty acid biosynthesis. In particular, the present invention is concerned with thiolactomycin analogues, compositions containing the same and the use thereof in inhibiting endoparasitic fatty acid biosynthesis.

Thiolactomycin is known to be an inhibitor of dissociable fatty acid synthases operable in plants and bacteria. Thiolactomycin has the following structure:

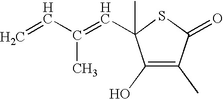

The three β-ketoacyl acyl carrier protein (ACP) synthases in peas are all inhibited by thiolactomycin. Condensing enzyme II (KAS-II) in peas, which catalyses the elongation of palmitoyl-ACP to stearoyl-ACP, is the most sensitive to inhibition by thiolactomycin. The short chain condensing enzyme (KAS III) in peas, which catalyses the initial condensation of acetyl-CoA with malonyl-ACP, is the least sensitive to inhibition by thiolactomycin.

A number of thiolactomycin derived compounds have also hitherto been prepared (for example as referenced in Biochemical Society Transactions, Vol 22 (1994), p.258) and their inhibition of fatty acid synthesis in peas investigated.

Racemic thiolactomycin is also known to inhibit fatty acid synthesis in Plasmodium falciparum. Plasmodium spp. (the causative agents of malaria) belong to the phylum Apicomplexa. An inhibitor(such as thiolactomycin)of fatty acid synthesis in parasites, such as Plasmodium falciparum, is potentially useful for therapeutic use in the treatment of Apicomplexan-mediated diseases, such as malaria, toxoplasmosis and the like.

Furthermore, racemic thiolactomycin has also been shown to inhibit fatty acid synthesis in Trypanosoma brucei with an IC₅₀ of approximately 150 μM [Morita, Y. S., Paul, K. S. and Englund, P. T. (2000). Specialised fatty acid synthesis in African Trypanosomes: myristate for GPI anchors. Science 288: 140-143].

We have now discovered, however, that certain compounds, in particular certain thiolactomycin analogues, are advantageous as inhibitors of at least one β-ketoacyl acyl carrier protein synthase operable in the fatty acid biosynthesis of endoparasites (such as endoparasites of the phylum Apicomplexa).

There is, therefore, provided by the present invention use of at least one compound, or pharmaceutically acceptable salt or prodrug thereof, as an inhibitor of at least one β-ketoacyl acyl carrier protein synthase operable in the fatty acid biosynthesis of endoparasites (such as endoparasites of the phylum Apicomplexa), which compound is of the following general formula (I)

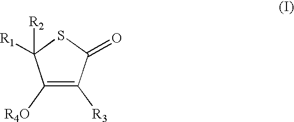

where

R₁ is selected from the group consisting of hydrogen, alkyl, (cyano)alkylene, alkenyl, alkynyl, (alkoxy)alkylene, (alkoxy)alkenylene, (alkoxy)alkynylene, cycloalkyl, (cycloalkyl)alkylene, (cycloalkyl)alkenylene, (cycloalkyl)alkynylene, heterocycle, (heterocycle)alkylene, (heterocycle)alkenylene, (heterocycle)alkynylene, aryl, (aryl)alkylene, (aryl)alkenylene, (aryl)alkynylene, (arylcarbonylarylene)alkylene, (arylcarbonylarylene) alkenylene and (arylcarbonylarylene)alkynylene;

R₂ is alkyl or cycloalkyl;

R₃ is alkyl or cycloalkyl; and

R₄ is hydrogen or alkyl; including racemic mixtures and enantiomers of said compound when the latter is chiral, but excluding the racemic mixture of a chiral compound of formula (I) in which R₁ is CH₂═CH—C(CH₃)═CH—, R₂ is methyl, R₃ is methyl and R₄ is hydrogen.

In the above formula, when R₄ is hydrogen, it will be appreciated that the compound is tautomeric. Of course, the tautomeric forms are both encompassed by the present invention.

In a preferred aspect of the present invention, however, there is provided use of at least one thiolactomycin analogue, or pharmaceutically acceptable salt or prodrug thereof, as an inhibitor of at least one β-ketoacyl acyl carrier protein synthase operable in the fatty acid biosynthesis of endoparasites (such as endoparasites of the phylum Apicomplexa), which thiolactomycin analogue is of general formula (I) substantially as hereinbefore described; including racemic mixtures and enantiomers of said thiolactomycin analogue when the latter is chiral; but excluding from formula (I) the racemic mixture and enantiomers of a chiral compound in which R₁ is CH₂═CH—C(CH₃)═CH—, R₂ is methyl, R₃ is methyl and R₄ is hydrogen.

Alkyl, alkenyl, alkynyl, alkylene, alkenylene and alkynylene substantially as hereinbefore described can be straight or branched (where appropriate)groups.

Typically, R₂ is C₁₋₆alkyl or C₃₋₆cycloalkyl and is suitably selected from the group consisting of methyl, ethyl, isopropyl and cyclopropyl. Preferably R₂ is methyl.

Typically, R₃ is C₁₋₆alkyl or C₃₋₆cycloalkyl and is suitably selected from the group consisting of methyl, ethyl, isopropyl and cyclopropyl. Preferably R₃ is methyl.

Typically, R₄ is hydrogen or C₁₋₆alkyl and is suitably selected from the group consisting of hydrogen and methyl. Preferably R₄ is hydrogen.

Typically R₁ is selected from the group consisting of hydrogen, C₁₋₂₀alkyl, (cyano)C₁₋₂₀ alkylene, C₂₋₂₀ alkenyl, C₂₋₂₀ alkynyl, (C₁₋₁₀alkoxy)C₁₋₂₀alkylene, (C₁₋₁₀alkoxy) C₂₋₂₀alkenylene, (C₁₋₁₀alkoxy) C₂₋₂₀alkynylene, C₃₋₈cycloalkyl, (C₃₋₈cycloalkyl) C₁₋₂₀alkylene, (C₃₋₈cycloalkyl) C₂₋₂₀alkenylene, (C₃₋₈cycloalkyl) C₂₋₂₀alkynylene, heterocycle, (heterocycle) C₁₋₂₀alkylene, (heterocycle) C₂₋₂₀alkenylene, (heterocycle) C₂₋₂₀alkynylene, aryl, (aryl) C₁₋₂₀alkylene, (aryl)C₂₋₂₀alkenylene, (aryl)C₂₋₂₀alkynylene, (arylcarbonylarylene) C₁₋₂₀alkylene, (arylcarbonylarylene) C₂₋₂₀alkenylene and (arylcarbonylarylene)C₂₋₂₀alkynylene.

Preferably, however, R₁ is selected from the group consisting of hydrogen, C₁₋₂₀alkyl, (cyano)C₁₋₂₀alkylene, C₂₋₂₀alkenyl, (C₁₋₁₀alkoxy) C₁₋₂₀alkylene, (heterocycle) C₁₋₂₀alkylene, (aryl) C₁₋₂₀alkylene, (aryl)C₂₋₂₀alkenylene and (arylcarbonylarylene) C₁₋₂₀alkylene. Suitably, heterocycle represents a 3 to 8 membered ring containing at least one heteroatom selected from oxygen, nitrogen and sulphur (preferably oxygen). A particularly preferred heterocycle is an epoxy ring. Suitably, aryl represents phenyl.

In the case where R₁ represents (cyano)C₁₋₂₀alkylene, it is preferred that R₁ is (cyano)C₁₋₆alkylene, especially (cyano)methylene. In a particular preferred aspect of the present invention, there is provided use of at least one thiolactomycin analogue, or pharmaceutically acceptable salt or prodrug thereof, as an inhibitor of at least one β-ketoacyl acyl carrier protein synthase operable in the fatty acid biosynthesis of endoparasites, which thiolactomycin analogue is of formula (IA)

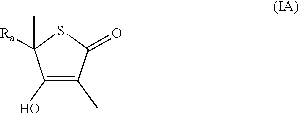

where Rₐ is (cyano)C₁₋₃alkylene.

As will be appreciated, thiolactomycin analogues of formula (IA) are a preferred subgroup of compounds of formula (I).

A further preferred subgroup of compounds of formula (I) is where R₁ in formula (I) represents hydrogen or an alkyl or alkenyl group of two to twenty, more suitably two to sixteen, carbon atoms, and may (where appropriate) comprise a straight or branched chain substantially as herein before described.

In a further preferred aspect of the present invention, therefore, there is provided use of at least one thiolactomycin analogue, or pharmaceutically acceptable salt or prodrug thereof, as an inhibitor of at least one β-ketoacyl acyl carrier protein synthase operable in the fatty acid biosynthesis of endoparasites, which thiolactomycin analogue is of formula (IB)

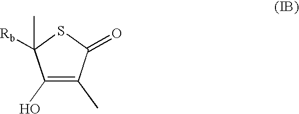

where R_{b} is hydrogen or C₃₋₁₂ alkyl.

As will be appreciated, thiolactomycin analogues of formula (IB) are a preferred subgroup of compounds of formula (I).

Suitably, R_{b} can be an alkyl group selected from the group consisting of CH₃(CH₂)₃—, (CH₃)₂CH(CH₂)₂—, CH₃(CH₂)₅—, CH₃(CH₂)₇—, CH₃(CH₂)₉—, (CH₃)₂CH (CH₂)₃CHCH₃ (CH₂)₂— and the like.

In a further preferred aspect of the present invention, there is provided use of at least one thiolactomycin analogue, or pharmaceutically acceptable salt or prodrug thereof, as an inhibitor of at least one β-ketoacyl acyl carrier protein synthase operable in the fatty acid biosynthesis of endoparasites, which thiolactomycin analogue is of formula (IC)

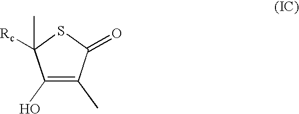

where R_{c} is C₂₋₁₆ alkenyl, but excluding the racemic mixture of a chiral compound of formula (IC) in which R_{c} is CH₂═CH—C(CH₃)═CH—.

As will be appreciated, thiolactomycin analogues of formula (IC) are also a preferred subgroup of compounds of formula (I).

Suitably R_{c} may (where appropriate)include more than one double bond and preferably can be selected from the group consisting of CH₂═CHCH₂—, (CH₃)₂C═CHCH₂—, (CH₃)₂C═CH(CH₂)₂CCH₃═CHCH₂—, (CH₃)₂C═CH (CH₂)₂C (CH₃)₂(CH₂)₂— and (CH₃)₂C═CH (CH₂)₂CCH₃—CH (CH₂)₂CCH₃═CHCH₂—.

In a further preferred aspect of the present invention, there is provided use of at least one thiolactomycin analogue, or pharmaceutically acceptable salt or prodrug thereof, as an inhibitor of at least one β-ketoacyl acyl carrier protein synthase operable in the fatty acid biosynthesis of endoparasites, which thiolactomycin analogue is of formula (ID)

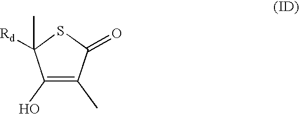

where R_{d} is (heterocycle)C₁₋₃alkylene.

Suitably R_{d} is (epoxy)C₁₋₃alkylene, preferably (epoxy)methylene.

As will be appreciated, thiolactomycin analogues of formula (ID) are also a preferred subgroup of thiolactomycin analogues of formula (I).

In a further preferred aspect of the present invention, there is provided use of at least one thiolactomycin analogue, or pharmaceutically acceptable salt or prodrug thereof, as an inhibitor of at least one β-ketoacyl acyl carrier protein synthase operable in the fatty acid biosynthesis of endoparasites, which thiolactomycin analogue is of formula (IE)

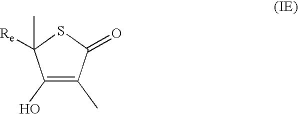

where Rₑ is (aryl)C₁₋₆alkylene or (aryl)C₂₋₆alkenylene.

Suitably, Rₑ is (phenyl)C₁₋₆alkylene or (phenyl)C₂₋₆alkenylene and can preferably be selected from the group consisting of benzyl, (phenyl)ethylene and (phenyl)propenylene.

As will be appreciated, thiolactomycin analogues of formula (IE) are also a preferred subgroup of compounds of formula (I).

In a further preferred aspect of the present invention, there is provided use of at least one thiolactomycin analogue, or pharmaceutically acceptable salt or prodrug thereof, as an inhibitor of at least one β-ketoacyl acyl carrier protein synthase operable in the fatty acid biosynthesis of endoparasites, which thiolactomycin analogue is of formula (IF)

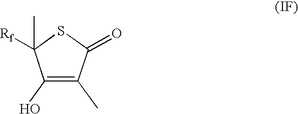

where R_{f} is (arylcarbonylarylene) C₁₋₆alkylene.

As will be appreciated, thiolactomycin analogues of formula (IF) are also a preferred subgroup of compounds of formula (I).

Suitably, R_{f} is (benzoylphenylene) C₁₋₆alkylene and preferably R_{f} can be (benzoylphenylene)methylene.

In a further preferred aspect of the present invention, there is provided use of at least one thiolactomycin analogue, or pharmaceutically acceptable salt or prodrug thereof, as an inhibitor of at least one β-ketoacyl acyl carrier protein synthase operable in the fatty acid biosynthesis of endoparasites, which thiolactomycin analogue is of formula (IG)

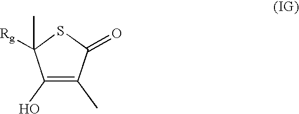

where R_{g} is (C₁₋₆alkoxy)C₁₋₆alkylene.

As will be appreciated, thiolactomycin analogues of formula (IG) are also a preferred subgroup of compounds of formula (I).

Suitably R_{g} is (C₁₃alkoxy)C₁₃alkylene and preferably is (ethoxy)ethylene.

Particularly preferably there is provided by the present invention use of at least one thiolactomycin analogue, or pharmaceutically acceptable salt or prodrug thereof, as an inhibitor of at least one β-ketoacyl acyl carrier protein synthase operable in the fatty acid biosynthesis of endoparasites, which thiolactomycin analogue is selected from compounds A to S as hereinafter described:

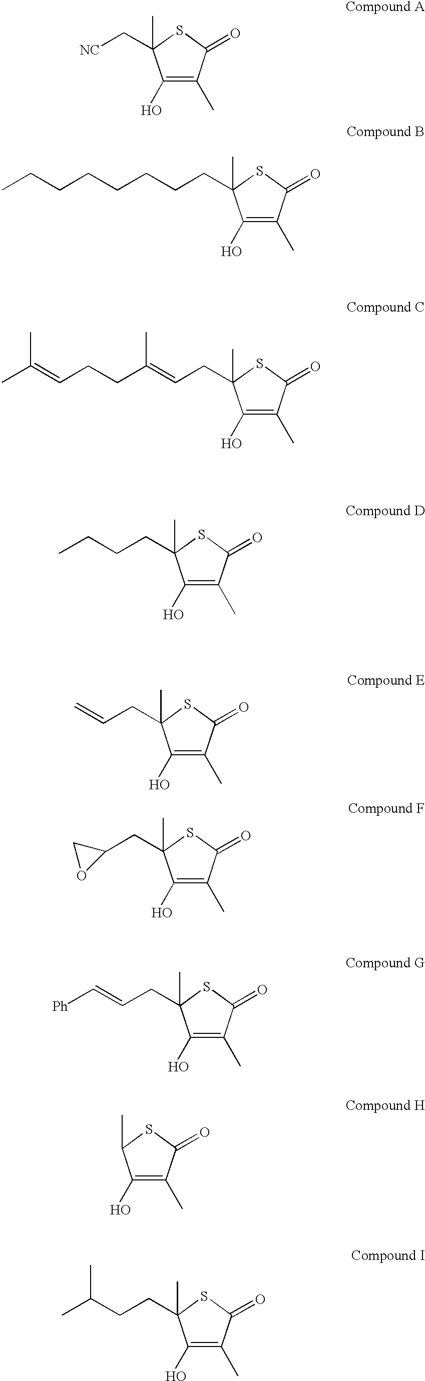

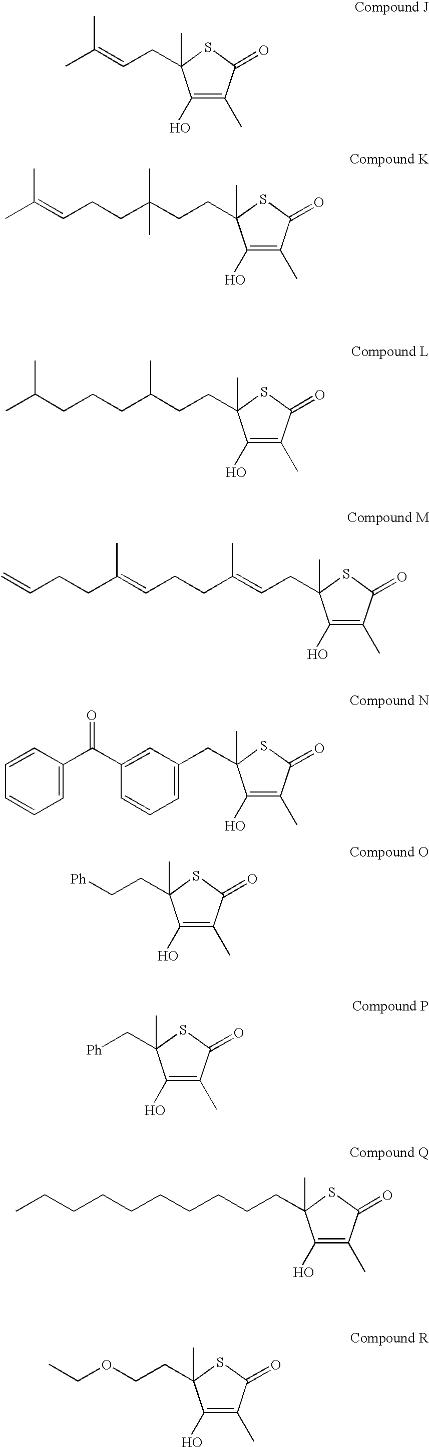

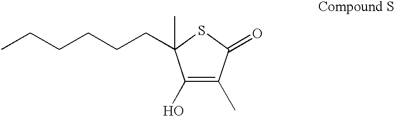

Compound C is the preferred thiolactomycin analogue for use in the present invention.

The above specified thiolactomycin analogues for use according to the present invention can be prepared by known synthetic techniques in the field of organic chemistry, for example as described in Tetrahedron Letters, Vol. 25, No. 46, pp5243-5246, 1984.

As indicated above, the present invention includes within its scope pharmaceutically acceptable salts of compounds of formula (I). The present invention further includes within its scope prodrugs or bioprecursors of compounds of formula (I) and in general such prodrugs will be substantially functional precursors of compounds of formula (I) which are readily convertible in vivo into the required active compound. Conventional procedures for selection and preparation of suitable prodrugs are well known in the art.

Chiral compounds of formula (I) according to the present invention may be prepared in racemic form, or as individual enantiomers that may be prepared by either enantiospecific synthesis or by resolution. Preferably, where chiral compounds of formula (I) according to the present invention are present as specific enantiomers, the enantiomer can be provided in substantially pure form, such as, for example, having an isomeric purity of at least about 95%. Such enantiomers include the (+) and (−) form of thiolactomycin itself, which enantiomers have not previously been suggested for inhibition of at least one β-ketoacyl acyl carrier protein synthase operable in the fatty acid biosynthesis of endoparasites.

Use according to the present invention of compounds of formula (I) substantially as hereinbefore described can preferably be directed to the inhibition of at least one β-ketoacyl acyl carrier protein synthase operable in the fatty acid biosynthesis of endoparasites of the phylum Apicomplexa.

The phylum Apicomplexa is a group of obligate endoparasites such as Plasmodium, including P. falciparum, P. vivax, P. malariae and P. ovale (causative agents of malaria); Eimeriida including Cryptosporidium sp. (often associated with diarrhoea and HIV in humans), Cyclospora sp. (often the cause of traveller's diarrhoea and also associated with HIV in humans), Eimeria sp. (often the cause of diseases in poultry and other animals), Sarcocystis sp. (often the cause of diseases in dogs and horses, such as toxoplasmosis or the like) and Toxoplasma gondii (the cause of toxoplasmosis in cats, which can be particularly problematic if HIV sufferers or pregnant women are infected thereby); Babesiidae including Babesiidae sp. (often the cause of diseases in cattle) and Theileriidae including Theileria sp. (again often the cause of diseases in cattle) and Cytauxzoon sp. (often the cause of a fatal cat disease particularly prevalent in USA).

Use according to the present invention of compounds of formula (I) substantially as hereinbefore described can also be directed to the inhibition of at least one β-ketoacyl acyl carrier protein synthase operable in the fatty acid biosynthesis of kinetoplastid flagellate endoparasites.

The kinetoplastid flagellate endoparasites include parasites of the genera Leishmania and Trypanosoma. Parasites in the genus Leishmania are the causative agents in humans of visceral leishmaniasis (Kala azar) and cutaneous leishmaniasis. Trypanosoma brucei gambiense and Trypanosoma brucei rhodesiense are the causative agents of sleeping sickness in humans, while Trypanosoma brucei brucei causes disease in domestic and wild animals. Trypanosoma cruzi is the causative agent of Chagas' disease, which is widespread in central and south America and can cause cardiac damage that can ultimately lead to death.

The term “endoparasite-mediated disease” as used herein denotes disease arising due to the presence of one or more endoparasites in a host animal, such as a human. The term “Apicomplexan-mediated disease” as used herein denotes disease arising due to the presence of one or more parasites of the phylum Apicomplexa (such as referred to above) in a host animal, such as a human.

While it is possible for compounds according to the present invention to be administered to a host animal as the substantially pure chemicals, it is preferable that these compounds are included in pharmaceutical compositions for administration to a host animal. There is, therefore, still further provided by the present invention a pharmaceutical composition comprising an inhibitory amount of at least one compound of formula (I), or pharmaceutically acceptable salt or prodrug thereof, substantially as herein before described, together with at least one acceptable carrier, diluent or excipient therefor. The carrier, diluent or excipient must be “acceptable” in the sense of being compatible with the other ingredients of the composition and not deleterious to the “host” animal.

Preferred compositions according to the present invention comprise at least one thiolactomycin analogue selected from compounds of formulae (IA), (IB), (IC), (ID),(IE), (IF) and (IG) substantially as herein before described. Most preferably, a composition according to the present invention comprises at least one thiolactomycin analogue selected from compounds A to S substantially as herein before described.

The term “inhibitory amount” as used herein denotes an amount of a compound substantially as hereinbefore described capable of substantially inhibiting at least one β-ketoacyl acyl carrier protein synthase operable in the fatty acid biosynthesis of endoparasites (particularly endoparasites of the phylum Apicomplexa) but having substantially no deleterious inhibitory effect on the fatty acid biosynthesis of an animal (particularly vertebral, even more particularly mammalian) host.

It will be appreciated, therefore, that the use of compounds according to the present invention can be of therapeutic value in the treatment of endoparasitic-mediated disease by effecting the substantial inhibition of the fatty acid biosynthesis of endoparasites but having substantially no deleterious inhibitory effect on the fatty acid biosynthesis of an animal (particularly vertebral, even more particularly mammalian) host.

A composition according to the present invention may further comprise at least one further therapeutic material effective in the treatment of endoparasite-mediated (particularly Apicomplexan-mediated) diseases.

The present invention can further provide a product comprising at least one compound of formula (I) substantially as herein before described and at least one further therapeutic material effective in the treatment of endoparasite-mediated disease, as a combined preparation for simultaneous, separate or sequential use in the treatment of endoparasite-mediated disease.

The further therapeutic material for use in a composition or product may comprise any material effective in the treatment of endoparasite-mediated disease and in a particular embodiment may comprise a further compound of formula (I). There is, therefore, further provided by the present invention a pharmaceutical composition comprising more than one compound of formula (I), or pharmaceutically acceptable salts or prodrugs thereof, substantially as herein before described, together with at least one acceptable carrier, diluent or excipient therefor. There is also provided a product comprising more than one compound of formula (I) (such as first and second compounds of formula (I)), or pharmaceutically acceptable salts or prodrugs thereof, as a combined preparation for simultaneous, separate or sequential use in the treatment of endoparasite-mediated disease.

Compositions or products according to the present invention include those suitable for oral, parenteral (including intravenous), rectal (including in particular administration by suppositories) and topical administration, although the most suitable route will generally depend upon the nature and condition of an animal host patient being treated and the specific endoparasite-mediated disease. The precise amount of a compound of formula (I) to be administered to a patient will depend upon a number of factors, including the age and sex of the patient, the specific endoparasite-mediated disease being treated and the route of administration substantially as described above.

Substantially as hereinbefore described, the present invention is particularly concerned with the treatment of Apicomplexan-mediated disease. Typically, the Apicomplexan-mediated disease to be treated according to the present invention can arise due to the presence of Plasmodium spp. in an animal host and as such the present invention is applicable for use in the treatment of malaria and related disease. Alternatively, the Apicomplexan-mediated disease can arise due to the presence of Eimeria sp. in an animal host.

There is further provided by the present invention use of at least one compound of formula (I) substantially as herein before described, in the manufacture of a medicament for the treatment of endoparasite-mediated (particularly Apicomplexan-mediated) disease. More particularly, there is provided use of at least one compound of formula (I) substantially as herein before described, in the manufacture of a medicament for the treatment of Apicomplexan-mediated disease arising due to the presence of Plasmodium spp. in an animal host. Even more particularly, the invention provides use of at least one compound of formula (I) substantially as herein before described in the manufacture of a medicament for use in the treatment of malaria. There is also provided by the present invention use of at least one compound of formula (I) substantially as herein before described, in the manufacture of a medicament for the treatment of Apicomplexan-mediated disease arising due to the presence of Eimeria sp. in an animal host.

There is also provided by the present invention a method of treating endoparasite-mediated (particularly Apicomplexan-mediated) disease in an animal host, which method comprises administering to the animal host an inhibitory amount of at least one compound of formula (I) substantially as herein before described.

The method of treatment according to the present invention can preferably be for use in the treatment of Apicomplexan-mediated disease arising due to the presence of Plasmodium spp. in the animal (in particular human) host, and as such can be particularly directed to the treatment of malaria and related disease. Alternatively, the method of treatment according to the present invention can be for use in the treatment of Apicomplexan-mediated disease arising due to the presence of Eimeria sp. in the animal host.

The present invention is also concerned with a method of inhibiting at least one β-ketoacyl acyl carrier protein synthase operable in the fatty acid biosynthesis of endoparasites (particularly endoparasites of the phylum Apicomplexa), which method comprises contacting the synthase with at least one compound of formula (I) so as to effect substantial inhibition of the synthase.

Preferably the synthase is operable in the fatty acid biosynthesis of Plasmodium spp. or Eimeria sp. substantially as herein before described.

The present invention will now be further illustrated by the following examples which do not limit the scope of the invention in any way.

### EXAMPLE 1

Plasmodium falciparum was maintained in human red blood cells and the inhibitory effects of racemic thiolactomycin, or compound C on its growth was measured, employing assay techniques as described by Makkler, M. T., Ries, J. M., Williams, J. A., Bancroft, J. E., Piper, R. C., Gibbins, B. L. and Hinrichs, D. J., Parasite lactate dehydrogenase as an assay for Plasmodium falciparum drug sensitivity. Am. J. Trop. Med. Hyg. 48, 739-41 (1993).

Racemic thiolactomycin exhibited a 50% inhibitory concentration (IC50) for parasite growth of 40 μM at 48 hours, whereas compound C exhibited a 50% inhibitory concentration (IC50) for parasite growth of 500 nM at 48 hours. Compound C was, therefore, approximately 80 times more active than racemic thiolactomycin in inhibiting growth of Plasmodium falciparum.

### EXAMPLE 2

Compound C was tested against Eimeria tenella parasites in two separate assay procedures, as described (i) in Inhibition of the development of Eimeria tenella in cultured bovine kidney cells by a soluble factor produced by peripheral blood lymphocytes from immune chickens, J. M. Bumstead, S. J. Topham, F. M. Tomley, Parasitology, 117.39-47 (1998) and (ii) Eimeria tenella: Parasite-specific incorporation of 3H Uracil as a quantitative measure of intracellular development, Schmatz, D. M., Crane, M. S., is Murray, P. K., Journal of Protozology (1986) 33:109-114.

Preincubation of compound C with parasites caused a significant reduction in invasion of Madin-Darby Bovine Kidney (MDBK) cells at concentrations of 10 μM and above.

In the second assay using infected MDBK cells, compound C caused a significant decrease in the uptake of uracil by the parasites (a recognised indicator of parasite viability) at concentrations greater than 5 μM.

1. Use of at least one compound, or pharmaceutically acceptable salt or prodrug thereof, as an inhibitor of at least one β-ketoacyl acyl carrier protein synthase operable in the fatty acid biosynthesis of endoparasites, which compound is of the following general formula (I) 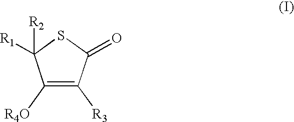
where
R₁ is selected from the group consisting of hydrogen, alkyl, (cyano)alkylene, alkenyl, alkynyl, (alkoxy)alkylene, (alkoxy)alkenylene, (alkoxy)alkynylene, cycloalkyl, (cycloalkyl)alkylene, (cycloalkyl)alkenylene, (cycloalkyl) alkynylene, heterocycle, (heterocycle) alkylene, (heterocycle)alkenylene, (heterocycle)alkynylene, aryl, (aryl)alkylene, (aryl)alkenylene, (aryl)alkynylene, (arylcarbonylarylene)alkylene (arylcarbonylarylene)alkenylene and (arylcarbonylarylene)alkynylene;
R₂ is alkyl or cycloalkyl;
R₃ is alkyl or cycloalkyl; and
R₄ is hydrogen or alkyl; including racemic mixtures and enantiomers of said compound when the latter is chiral, but excluding the racemic mixture of a chiral compound of formula (I) in which R₁ is CH₂═CH—C(CH₃)═CH—, R₂ is methyl, R₃ is methyl and R₄ is hydrogen.

2. Use of at least one thiolactomycin analogue, or pharmaceutically acceptable salt or prodrug thereof, as an inhibitor of at least one β-ketoacyl acyl carrier protein synthase operable in the fatty acid biosynthesis of endoparasites, which thiolactomycin analogue is of general formula (I) 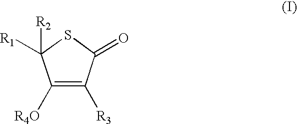
where
R₁ is selected from the group consisting of hydrogen, alkyl, (cyano)alkylene, alkenyl, alkynyl, (alkoxy)alkylene, (alkoxy)alkenylene, (alkoxy)alkynylene, cycloalkyl, (cycloalkyl)alkylene, (cycloalkyl)alkenylene, (cycloalkyl)alkynylene, heterocycle, (heterocycle)alkylene, (heterocycle)alkenylene, (heterocycle)alkynylene, aryl, (aryl)alkylene, (aryl)alkenylene, (aryl)alkynylene, (arylcarbonylarylene)alkylene, (arylcarbonylarylene)alkenylene (arylcarbonylarylene)alkenylene;
R₂ is alkyl or cycloalkyl;
R₃ is alkyl or cycloalkyl; and
R₄ is hydrogen or alkyl; including racemic mixtures and enantiomers of said thiolactomycin analogue when the latter is chiral but excluding from formula (I) the racemic mixture and enantiomers of a chiral compound in which R₁ is CH₂═CH—C(CH₃)═CH—, R₂ is methyl, R₃ is methyl and R₄ is hydrogen.

3. Use according to claim 1 or 2, wherein R₂ is C₁,alkyl or C₃₋₆cycloalkyl.

4. Use according to claim 3, wherein R₂ is selected from the group consisting of methyl, ethyl, isopropyl and cyclopropyl.

5. Use according to claim 4, wherein R₂ is methyl.

6. Use according to any preceding claim, wherein R₃ is C₁₋₆ alkyl or C₃₋₆cycloalkyl.

7. Use according to claim 6, wherein R₃ is selected from the group consisting of methyl, ethyl, isopropyl and cyclopropyl.

8. Use according to claim 7, wherein R₃ is methyl.

9. Use according to any preceding claim, wherein R₄ is hydrogen or C₁₋₆alkyl.

10. Use according to claim 9, wherein R₄ is selected from the group consisting of hydrogen and methyl.

11. Use according to claim 10, wherein R₄ is hydrogen.

12. Use according to any preceding claim, wherein R₁ is selected from the group consisting of hydrogen, C₁₋₂₀alkyl, (cyano)C₁₋₂₀ alkylene, C₂₋₂₀ alkenyl, C₂₋₂₀ alkynyl, (C₁₋₁₀alkoxy)C₁₋₂₀alkylene, (C₁₋₁₀alkoxy) C₂₋₂₀alkenylene, (C₁₋₁₀alkoxy) C₂₋₂₀alkynylene, C₃₋₈cycloalkyl, (C₃₋₆cycloalkyl) C₁₋₂₀alkylene, (C₃₋₈cycloalkyl) C₂₋₂₀alkenylene, (C₃₋₈cycloalkyl)C₂₋₂₀alkynylene, heterocycle, (heterocycle) C₁₋₂₀alkylene, (heterocycle) C₂₋₂₀alkenylene, (heterocycle) C₂₋₂₀alkynylene, aryl, (aryl) C₁₋₂₀alkylene, (aryl)C₂₋₂₀alkenylene, (aryl)C₂₋₂₀alkynylene, (arylcarbonylarylene) C₁₋₂₀alkylene, (arylcarbonylarylene)C₂₋₂₀alkenylene and (arylcarbonylarylene) C₂₋₂₀alkynylene.

13. Use according to claim 12, wherein R₁ is selected from the group consisting of hydrogen, C₁₋₂₀alkyl, (cyano)C₁₋₂₀alkylene, C₂₋₂₀alkenyl, (C₁₋₁₀alkoxy) C₁₋₂₀alkylene, (heterocycle) C₁₋₂₀alkylene, (aryl) C₁₋₂₀alkylene, (aryl) C₂₋₂₀alkenylene and (arylcarbonylarylene) C₁₋₂₀alkylene.

14. Use according to claim 13, wherein R₁ is selected from the group consisting of hydrogen, C₃₋₁₂alkyl, (cyano) C₁₋₃alkylene, C₂₋₁₆alkenyl, (C₁₋₆alkoxy) C₁₋₆alkylene, (heterocycle) C₁₋₃alkylene, (aryl)C₁₋₆alkylene, (aryl)C₂₋₆alkenylene and (arylcarbonylarylene)C₁₋₆alkylene.

15. Use according to any preceding claim, wherein heterocycle represents a 3 to 8 membered ring containing at least one heteroatom selected from oxygen, nitrogen and sulphur.

16. Use according to claim 15, wherein the heterocycle is an epoxy ring.

17. Use according to any preceding claim, wherein aryl represents phenyl.

18. Use according to any of claims 13 to 17, wherein R₁ is selected from the group consisting of hydrogen, C₃₋₁₂alkyl, (cyano)C₁₋₃alkylene, C₂₋₁₆alkenyl, (C₁₋₃alkoxy)C₁₋₃alkylene, (epoxy)C₁₋₃alkylene, (phenyl)C₁₋₆alkylene, (phenyl)C₂₋₆alkenylene and (benzoylphenylene)C₁₋₆alkylene.

19. Use according to claim 18, wherein R₁ is selected from the group consisting of hydrogen, CH₃(CH₂)₃—, (CH₃)₂CH(CH₂)₂—, CH₃(CH₂)₅—, CH₃(CH₂)₇—, CH₃(CH₂)₉—, (CH₃)₂CH(CH₂)₃CHCH₃(CH₂)₂—, cyanomethylene, CH₂═CHCH₂—, (CH₃)₂═CHCH₂—, (CH₃)₂C═CH (CH₂)₂CCH₃═CHCH₂—, (CH₃)₂C═CH(CH₂)₂C(CH₃)₂(CH₂)₂—, (CH₃)₂C═CH (CH₂)₂CCH₃═CH (CH₂)₂CCH₃═CHCH₂—, (ethoxy)ethylene, (epoxy)methylene, benzyl, (phenyl)ethylene, (phenyl)propenylene and (benzoylphenylene)methylene.

20. Use of at least one thiolactomycin analogue, or pharmaceutically acceptable salt or prodrug thereof, as an inhibitor of at least one β-ketoacyl acyl carrier protein synthase operable in the fatty acid biosynthesis of endoparasites, which thiolactomycin analogue is of formula (IA) 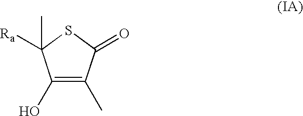
where Rₐ is (cyano)C₁₋₃alkylene.

21. Use according to claim 20, wherein Rₐ represents (cyano)methylene.

22. Use of at least one thiolactomycin analogue, or pharmaceutically acceptable salt or prodrug thereof, as an inhibitor of at least one β-ketoacyl acyl carrier protein synthase operable in the fatty acid biosynthesis of endoparasites, which thiolactomycin analogue is of formula (IB) 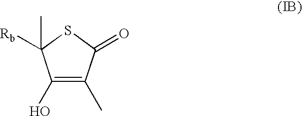
where R_{b} is hydrogen or C₃₋₁₂alkyl.

23. Use according to claim 22, wherein R_{b} is selected from the group consisting of CH₃(CH₂)₃—, (CH₃)₂CH(CH₂)₂—, CH₃(CH₂)₅—, CH₃(CH₂)₇—, CH₃(CH₂)₉— and (CH₃)₂CH(CH₂)₃CHCH₃(CH₂)₂—.

24. Use of at least one thiclactomycin analogue, or pharmaceutically acceptable salt or prodrug thereof, as an inhibitor of at least one β-ketoacyl acyl carrier protein synthase operable in the fatty acid biosynthesis of endoparasites, which thiolactomycin analogue is of formula (IC) 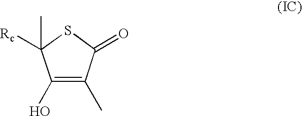
where R_{c} is C₂₋₁₆ alkenyl, but excluding the racemic mixture of a chiral compound of formula (IC) in which R_{c} is CH₂═CH—C(CH₃)═CH—.

25. Use according to claim 24, wherein R_{c} is selected from the group consisting of CH₂═CHCH₂—, (CH₃)₂C═CHCH₂—, (CH₃)₂C═CH(CH₂)₂CCH₃═CHCH₂—, (CH₃)₂C═CH(CH₂)₂C(CH₃)₂(CH₂)₂— and (CH₃)₂C═CH(CH₂)₂CCH₃═CH(CH₂)₂CCH₃═CHCH₂—.

26. Use of at least one thiolactomycin analogue, or pharmaceutically acceptable salt or prodrug thereof, as an inhibitor of at least one β-ketoacyl acyl carrier protein synthase operable in the fatty acid biosynthesis of endoparasites, which thiolactomycin analogue is of formula (ID) 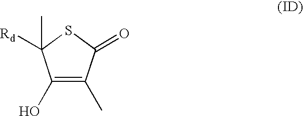
where R_{d} is (heterocycle)C₁₋₃alkylene.

27. Use according to claim 26, wherein R_{d} is (epoxy) C₁₋₃alkylene.

28. Use according to claim 27, wherein R_{d} is (epoxy)methylene.

29. Use of at least one thiolactomycin analogue, or pharmaceutically acceptable salt or prodrug thereof, as an inhibitor of at least one β-ketoacyl acyl carrier protein synthase operable in the fatty acid biosynthesis of endoparasites, which thiolactomycin analogue is of formula (IE) 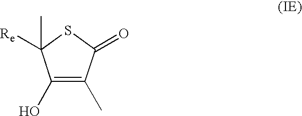
where Rₑ is (aryl)C₁₋₆alkylene or (aryl)C₂₋₆alkenylene.

30. Use according to claim 29, wherein Rₑ is (phenyl)C₁₋₆alkylene or (phenyl)C₂₋₆alkenylene.

31. Use according to claim 30, wherein Rₑ is selected from the group consisting of benzyl, (phenyl)ethylene and (phenyl)propenylene.

32. Use of at least one thiolactomycin analogue, or pharmaceutically acceptable salt or prodrug thereof, as an inhibitor of at least one β-ketoacyl acyl carrier protein synthase operable in the fatty acid biosynthesis of endoparasites, which thiolactomycin analogue is of formula (IF) 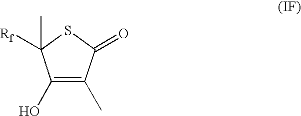
where R_{f} is (arylcarbonylarylene)C₁₋₆alkylene.

33. Use according to claim 32, wherein R_{f} is (benzoylphenylene) C₁₋₆alkylene.

34. Use according to claim 33, wherein R_{f} is (benzoylphenylene)methylene.

35. Use of at least one thiolactomycin analogue, or pharmaceutically acceptable salt or prodrug thereof, as an inhibitor of at least one β-ketoacyl acyl carrier protein synthase operable in the fatty acid biosynthesis of endoparasites, which thiolactomycin analogue is of formula (IG) 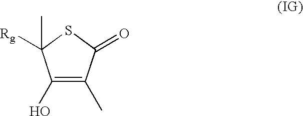
where R_{g} is (C₁₋₆alkoxy)C₁₋₆alkylene.

36. Use according to claim 35, wherein R_{g} is (C₁₋₃alkoxy) C₁₋₃alkylene.

37. Use according to claim 36, wherein R_{g} is (ethoxy)ethylene.

38. Use of at least one thiolactomycin analogue, or pharmaceutically acceptable salt or prodrug thereof, as an inhibitor of at least one β-ketoacyl acyl carrier protein synthase operable in the fatty acid biosynthesis of endoparasites, which thiolactomycin analogue is selected from compounds A to S:

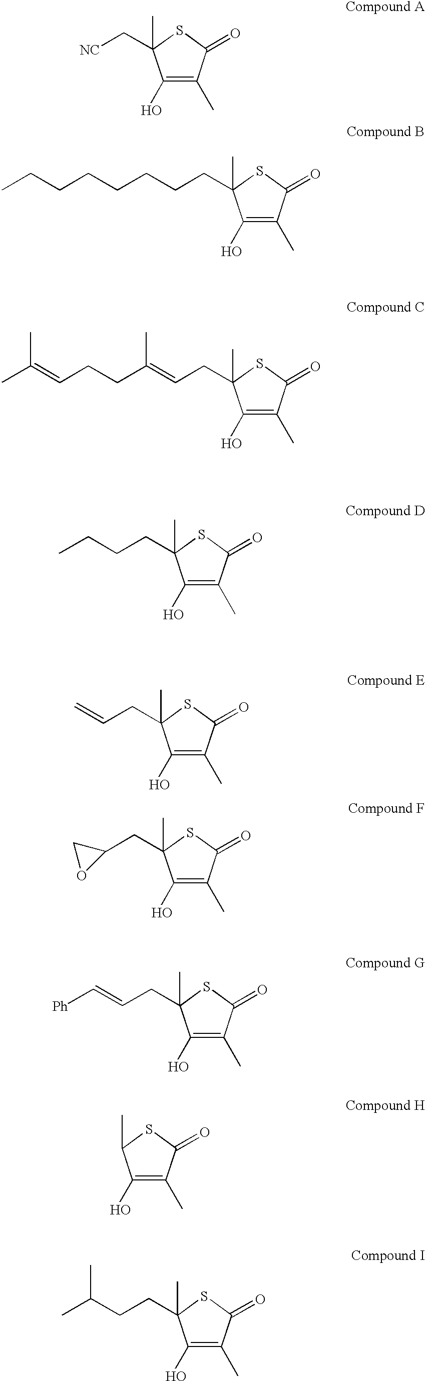

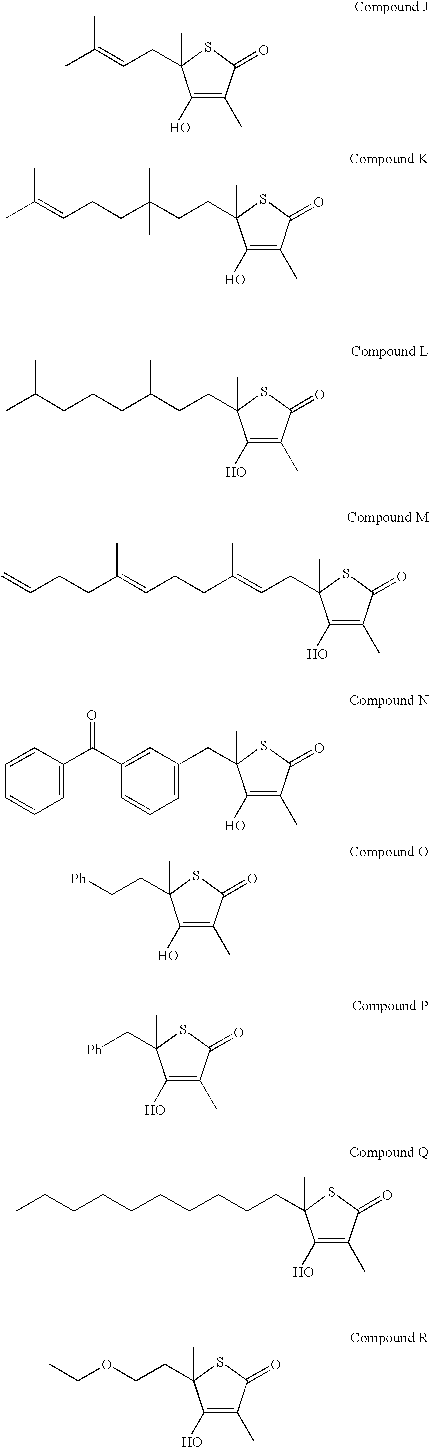

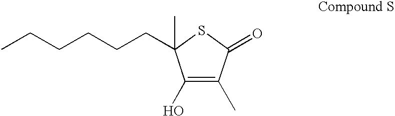

39. Use according to any preceding claim, wherein the endoparasites are of the phylum Apicomplexa.

40. Use according to claim 39, wherein the endoparasites are Plasmodium endoparasites.

41. Use according to claim 39, wherein the endoparasites are Eimeria endoparasites.

42. A pharmaceutical composition comprising an inhibitory amount of at least one compound as defined in any of claims 1 to 38, or pharmaceutically acceptable salt or prodrug thereof, together with at least one acceptable carrier, diluent or excipient therefor.

43. A composition according to claim 42, wherein said compound is selected from thiolactomycin analogues of formula (1A) as defined in claims 20 or 21; or thiolactomycin analogues of formula (1B) as defined in claims 22 or 23; or thiolactomycin analogues of formula (1C) as defined in claims 24 or 25; or thiolactomycin analogues of formula (ID) as defined in claims 26 to 28; or thiolactomycin analogues of formula (IE) as defined in claims 29 to 31; or thiolactomycin analogues of formula (IF) as defined in claims 32 to 34; or thiolactomycin analogues of formula (IG) as defined in claims 35 to 37.

44. A composition according to claim 43, wherein said thiolactomycin analogue is selected from compounds A to S as defined in claim 38.

45. A composition according to any of claims 42 to 44, which further comprises at least one further therapeutic material effective in the treatment of endoparasite-mediated disease.

46. A product comprising at least one compound as defined in any of claims 1 to 38 and at least one further therapeutic material effective in the treatment of endoparasite-mediated disease, as a combined preparation for simultaneous, separate or sequential use in the treatment of endoparasite-mediated disease.

47. Use of at least one compound as defined in any of claims 1 to 38, in the manufacture of a medicament for the treatment of endoparasite-mediated disease.

48. Use according to claim 47, in the manufacture of a medicament for the treatment of Apicomplexan-mediated disease arising due to the presence of Plasmodium spp. in an animal host.

49. Use according to claim 48, in the manufacture of a medicament for use in the treatment of malaria.

50. Use according to claim 47, in the manufacture of a medicament for the treatment of Apicomplexan-mediated disease arising due to the presence of Eimeria sp. in an animal host.

51. A method of treating endoparasite-mediated disease in an animal host, which method comprises administering to the animal host an inhibitory amount of at least one compound as defined in any of claims 1 to 38.

52. A method according to claim 51, which method comprises treating Apicomplexan-mediated disease arising due to the presence of Plasmodium spp. in the animal host.

53. A method according to claim 52, which method comprises treating malaria in the animal host.

54. A method according to claim 51, which method comprises treating Apicomplexan-mediated disease arising due to the presence of Eimeria sp. in the animal host.

55. A method of inhibiting at least one β-ketoacyl acyl carrier protein synthase operable in the fatty acid biosynthesis of endoparasites, which method comprises contacting the synthase with at least one compound as defined in any of claims 1 to 38 so as to effect substantial inhibition of the synthase.
